(19) United States
(12) Patent Application Publication
(10) Pub. No.: US 2010/0268657 A1
(43) Pub. Date: Oct. 21, 2010

(54) SYSTEM AND METHOD FOR TRACKING AND MANAGING TRANSPORTATION OF SPECIMENS

(76) Inventor: H. Davis Taylor, Florence, SC (US)

Correspondence Address: NEXSEN PRUET, LLC, P.O. BOX 10648, GREENVILLE, SC, 29603, US

(21) Appl. No.: 12/534,199

(22) Filed: Aug. 3, 2009

Related U.S. Application Data
(62) Division of application No. 11/368,819, filed on Mar. 6, 2006.
(60) Provisional application No. 60/658,533, filed on Mar. 4, 2005.

(51) Int. Cl. G06Q 30/00

(52) U.S. Cl. 705/333

(57) ABSTRACT

This invention is a system and method for tracking and managing transportation of specimens from a first location such as a medical office to a second location such as a testing lab for processing. A courier using a portable computing device can receive information representing that a specimen is ready for pick up from a first location. The courier will travel to a first location and enter specimen identification information into the portable computing device as well as first location information. Therefore, the portable computing device will have a record that the specimen has been picked up from the first location. The specimen is then carried to a second location and the courier enters the status of the specimen to show that it has been delivered to the second location for processing and a record exists to show that the specimen has been delivered to the second location. Lab results can be sent via the invention from the second location to the first location. A chain of custody and tracking system is therefore provided to reduce the risk of mishandled or even lost specimens and processing results.

### CLAIM OF PRIORITY

This application claims priority to U.S. Provisional patent Application, Ser. No. 60/658,533 entitled SYSTEM AND METHOD FOR TRACKING AND MANAGING TRANSPORTATION OF MEDICAL SPECIMENS, filed Mar. 4, 2005, herein incorporated by reference.

### FIELD OF THE INVENTION

This invention is directed to a system and method for tracking and managing the transportation of specimens such as medical specimens. More particularly, this invention is directed to a system and method for using a computer system for scanning, tracking and maintaining chain of custody of medical specimens from original source locations (first locations) to lab locations (second locations) for processing.

### BACKGROUND OF THE INVENTION

Medical specimens are used in a variety of ways. While the most common medical specimens are collected for health care at doctors' offices and hospitals, specimens are also collected for such applications as life insurance qualification, drug testing for employees and athletes, criminal investigation, and other forensic investigations. Medical specimens can be collected from humans as well as animals.

Traditionally, once a medical specimen is collected, the medical couriers transport the medical specimens from the collection source to a testing or processing lab. To maintain the chain of custody of the medical specimen and preserve the integrity of the test results, the possession of the medical specimen by the medical courier must be tracked and accounted for. Once the medical specimen is collected, the specimen can be associated with information from the collection source or first location. Such information can include patient number, location address, location number, crime scene number, or other such location identification. The specimen can have identification information that can be through the use of a barcode placed on the medical specimen container. The specimen is then transported to a second location such as a lab for processing or testing so that test results can be compiled based upon the need of those requesting the testing.

The results from testing of medical specimens can extraordinarily affect the source of the medical specimen. For example, the test results can be used to diagnose disease, to clear or convict a defendant in a criminal investigation, to determine whether employment may or may not be terminated and to determine eligibility for athletics. Significant problems arise when a medical specimen is lost, improperly identified, or improperly collected. Further, if the test results are not returned to the collection location, significant issues can arise. Improper identification, tracking, chain of custody and failure to return test result can lead to legal liability for the test requesting entity, the lab or the medical specimen courier. Further, such errors can result in improper criminal convictions, lack of criminal convictions, wrongful employment rejections or termination, tort liability for the medical specimen collector, lab or medical courier, and defamation of the medical specimen source. Imagine the liability incurred when an employee is terminated based upon a positive drug test due to test results from a medical specimen that was not collected from that employee. Further, imagine the liability were an employee addicted to drugs allowed to continue in a sensitive employment position such as child day care because the medical specimen for drug testing was lost.

Therefore, it is critical that the medical specimen identification be properly associated with the source, tracked, and a proper chain of custody maintained. It would be advantageous to provide for tracking reports and chain of custody reports that would show proper chain of custody, status of the lab testing, lab testing result information such as delivery date and date testing was preformed and other such management and administration desired by those skilled in the art.

Further, the medical courier can travel to many locations in a given week. Therefore, it would be advantageous that since the medical courier must routinely travel to the collection source and lab, that the medical courier can also track, maintain and stock inventory of supplier for the collection source or the lab.

DESCRIPTION OF THE DRAWINGS

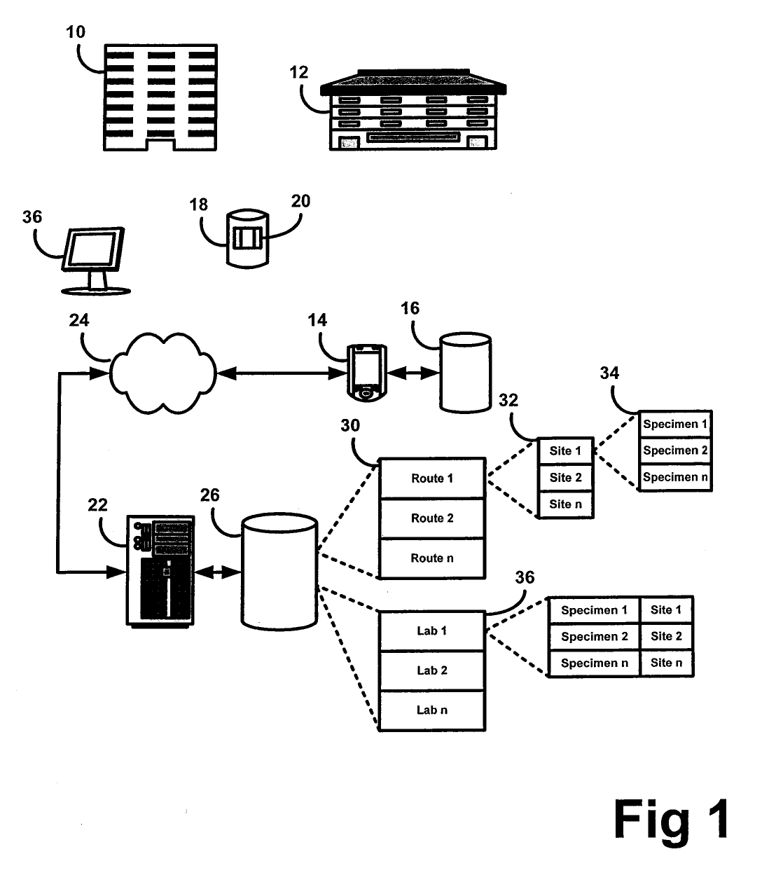

FIG. 1 is a schematic of component of the invention;

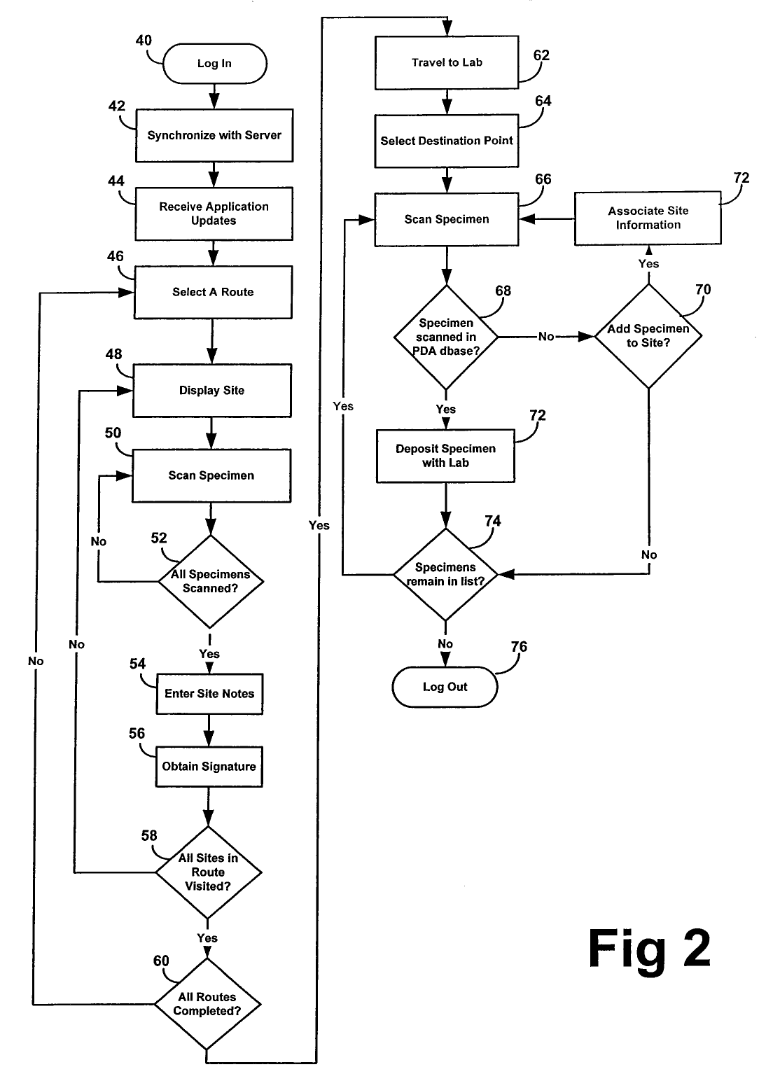

FIG. 2 is a flow chart of the operation of the invention; and,

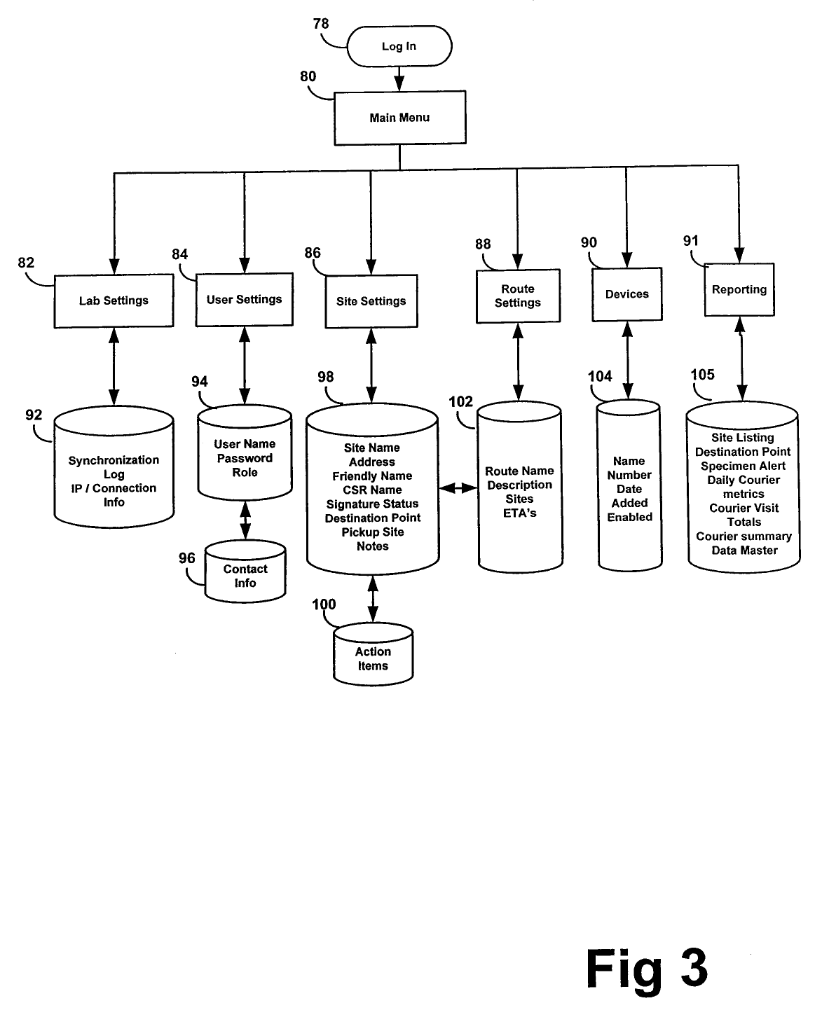

FIG. 3 is a schematic of the operation of the PDA used with the invention.

### DETAILED DESCRIPTION OF THE INVENTION

The detailed description that follows may be presented in terms of program procedures executed on a computer or network of computers. These procedural descriptions are representations used by those skilled in the art to most effectively convey the substance of their work to others skilled in the art. These procedures herein described are generally a self-consistent sequence of steps leading to a desired result. These steps require physical manipulations of physical quantities such as electrical or magnetic signals capable of being stored, transferred, combined, compared, or otherwise manipulated readable medium that is designed to perform a specific task or tasks. Actual computer or executable code or computer readable code may not be contained within one file or one storage medium but may span several computers or storage mediums. The terms “host” and “server” may be hardware, software, or combination of hardware and software that provides the functionality described herein. This invention thereby allows multiple users, being geographically dispersed, to interact with data relating to physical characteristics of manufactured products using a system that ensures the precise and accurate conveyance of such information.

The present invention is described below with reference to flowchart illustrations of methods, apparatus (“systems”) and computer program products according to the invention. It will be understood that each block of a flowchart illustration can be implemented by a set of computer readable instructions or code. These computer readable instructions may be loaded onto a general purpose computer, special purpose computer, or other programmable data processing apparatus to produce a machine such that the instructions will execute on a computer or other data processing apparatus to create a means for implementing the functions specified in the flowchart block or blocks.

These computer readable instructions may also be stored in a computer readable medium that can direct a computer or other programmable data processing apparatus to function in a particular manner, such that the instructions stored in a computer readable medium produce an article of manufacture including instruction means that implement the functions specified in the flowchart block or blocks. Computer program instructions may also be loaded onto a computer or other programmable apparatus to produce a computer executed process such that the instructions are executed on the computer or other programmable apparatus providing steps for implementing the functions specified in the flowchart block or blocks. Accordingly, elements of the flowchart support combinations of means for performing the special functions, combination of steps for performing the specified functions and program instruction means for performing the specified functions. It will be understood that each block of the flowchart illustrations can be implemented by special purpose hardware based computer systems that perform the specified functions, or steps, or combinations of special purpose hardware or computer instructions. The present invention is now described more fully herein with reference to the drawings in which the preferred embodiment of the invention is shown. This invention may, however, be embodied in many different forms and should not be construed as limited to the embodiment set forth herein. Rather, these embodiments are provided so that this disclosure will be thorough and complete and will fully convey the scope of the invention to those skilled in the art.

Referring now to FIG. 1, a collection location or first location 10 is shown where medical specimens are collected for a variety of uses. A medical specimen, upon a request to be tested, is then transported to a second location such as a lab 12 for processing such as testing. A medical courier arrives at collection location 10 with a portable computing device 14 having a portable computer readable medium 16. The medical specimen 18 is associated with specimen identification information 20 which can be entered, such as by scanning a barcode, in the portable device 14 so that the medical specimen identification information is stored in portable computer readable medium 16. Identification information 20 may be a bar code, a RFID tag, or any other known identifier. Additionally, the collection point information, or first location process or status information, can be stored in portable computer readable medium 16 and associated with the medical specimen.

Other such information that can be collected upon the portable device receiving the medical specimen identification marking can include process or status information which can include date, time, medical courier identification, individual providing the specimen, location of the site where the specimen was retrieved, and other such information useful for maintaining a chain of custody of the medical specimen.

Once the medical specimen is retrieved from the collection location 10, it is transported to a second location such as testing facility 12 so that requested processing can be performed on the medical specimen. The medical specimen is then transported to testing location 12 by a medical courier who has portable device 14. Upon delivery of the medical specimen to the testing location, the specimen identification information is entered into portable device 14 so that portable computer readable medium 16 contains a record of the medical specimen being delivered to testing location 12. Additionally, processing information can be associated with the delivery of the medical specimen to the testing location such as date, time, person who has received the medical specimen at testing location, and medical courier identification.

To facilitate the task of providing medical courier services, a route that the medical courier must travel can be uploaded to portable device 14 from a central server 22 via network 24. Portable device 14 can be in electronic communication with server 22 by such means as radio frequency, infrared, or wired communications such as through Internet network connections, or other such communication means. Since portable device 14 can be in electronic communication with server 22, information contained on the server's computer readable medium 26 can be transmitted to portable device 14 and stored in portable computer readable medium 16. Additionally, information from portable device 14 can be transmitted to server 22 and stored in the server's computer readable medium 26.

In one embodiment, information on server 22 can include the route information representing the path that the medical courier is to travel during a particular period of time. There can be one, two, or as many routes as the user wishes to designate transmitted to the portable device. By transmitting route information to the portable device, the medical courier can be provided with a route to be taken during the day. Full route information can be stored on computer readable medium 26 and is designated as 30 in FIG. 1. For each route, there can be one or more sites, designated as 32, associated with the route. For example, collection site 1 may be part of route 1 as well as collection site 2, shown as 32a. Therefore, route 1, when reviewed by the user, would show that site 1 is to be visited, and then site 2 is to be visited.

For each site, the medical courier retrieves each medical specimen that is present at the particular site and the medical specimen identification information for each specimen is associated with that site. The information associated with each specimen is shown as 34. Therefore, once the medical courier retrieves specimens from each site on each route, computer readable medium 16 can store process information such as route information 30, site information 32, and specimen information 34 reflecting the specimens that were collected, the site where the specimen was collected from, and the route where the site belongs. This information can be transmitted to server 22 and stored on the server's computer readable medium 26.

It is important to note that a route may also contain labs so that during a route, specimens can be dropped off at a particular lab, even between stops at specific sites. Lab information, shown as 36, can be stored in computer readable medium 26 or computer readable medium 16. When the medical courier travels to a lab 12, the specimens that are to be delivered to that lab are entered into the portable device 14 according to identification 20 and designated as delivered to lab 12. Therefore, the process of entering the identification information of a medical specimen allows the portable device to show which specimens are in the custody of the medical courier and which have been delivered to a lab for processing as well as process information such as time, date, location, and the individual who received the medical specimen. The lab can also be provided with the specimen information 34 and associated site information 32 when the medical specimen is delivered to the lab location 12.

In one embodiment, a GPS device is associated with portable device 14 so that when a medical specimen is retrieved from a first location or depository lab location, the location information can be associated with the transaction of receipt or delivery and stored in association with that particular medical specimen. The GPS device may be incorporated with portable device 14, or may be a stand alone device.

Portable device 14 can be “synchronized” with server 22 so that route information, site information, process information, status information, and lab information can be transmitted to portable device 14 and stored in portable device computer readable medium 16. Additionally, other information can also be transmitted to portable device 14 to assist in the administration and management of medical specimen transport. Such information can include users of the portable device, passwords, contact information for collection locations and lab locations, software update information transmitted from server 22 to portable device 14. Further, comments or notes associated with each route or site or lab can also be transmitted from server 22 to portable device 14 as well as from portable device 14 to server 22.

First location information can be specific to a site and can include the estimated time of arrival (ETA). This can be received from server 22 so that collection site 10 can inform the medical courier via portable device 14 that it requests medical specimen pickup at a particular time. In one embodiment, collection site 10 can access server 22 through client 36 and provide server 22 with a preferred pickup time so that server 22 can provide such information to the medical courier through portable device 14.

In one embodiment, the identification 20 of a medical specimen is a barcode and portable device 14 contains a barcode scanner for receiving the identification mark of the medical specimen.

When the medical courier is retrieving a medical specimen at site 10, site visit notes can be entered into portable device 14, stored in computer readable medium 16, transmitted to server 22, and stored in server computer readable medium 26. The site visit notes can be entered through alphanumeric keyboards or can be selected from predetermined site notes that will then be stored in portable device 14.

A signature can be obtained at a collection site 10 and entered into portable device 14 and stored in computer readable medium 16 as part of the process information. In one embodiment, once the signature is entered into portable device 14, subsequent retrieval of medical specimens from that particular collection site may be restricted. Therefore, the signature can represent such circumstances as collection site 10 verified that medical specimens were retrieved and the medical courier has completed receipt of all medical specimens to be picked up at that site.

When lab location 12 has medical specimens dropped off, identification mark 20 for each medical specimen is entered into portable device 14. Once the information is entered, the specimen can be removed from a drop-off list which, when such list is empty, indicates that each specimen to be delivered to that particular lab location has been delivered to the lab location and no specimens in the custody of the medical courier need to be dropped off. In the event that medical specimen information exists in the list associated with that particular lab location, the medical courier is informed that further specimens need to be dropped at that lab location.

Server 22 can contain an application for managing information and storing in computer readable medium 26. For example, a user interface can be used to maintain route information, site information, and lab information on server 22.

Referring now to FIG. 2, the operation of the invention will be described in more detail. In step 40, the user of portable device 14 logs in. The portable device can then be synchronized with server 22 in step 42. Portable device can then receive application updates in step 44, if any are present on server 22. In step 46, the user selects a route to travel which can include collection locations or lab locations. A pre-selected site according to the route is displayed for visiting at step 48, once the medical courier arrives at the selected site, a specimen can be entered into portable device 14, such as by scanning, at step 50. If there still remains specimens to be retrieved and scanned at step 52, the process returns to step 50 and continues.

When all specimens are scanned, site notes can be entered concerning a particular site at step 54 and a signature may be obtained at step 56 so that the medical courier is allowed to move on to the next site. A determination is made at step 58 as to whether all sites have been visited within the route. If not, the process returns to step 48 and begins again. If so, a determination is made at step 60 as to whether all routes are complete. If not, the process returns to step 46 for the next round, otherwise, the medical courier may travel to a lab at step 62. Upon reaching the particular destination point for the lab at step 64, the specimen to be deposited at the lab is scanned at step 66. A determination is made as to whether the scanned specimen exists in portable computer readable medium 16 of portable device 14. If not, the determination is made at step 70 as to whether to associate the scanned specimen that was not discovered in portable computer readable medium 16 with the specific site at step 70. Determination is made then in association of the site to the scanned specimen is made at step 72 and the process returns to step 66. If the determination at step 68 finds the specimen in computer readable medium 16, then the specimen is deposited with the lab location at step 72. The determination is made at step 74 as to whether there are specimens remaining in computer readable medium of portable device 14. If so, the process returns to step 66. If all specimens have been scanned and delivered to those lab locations, the user can log out at step 76.

Referring now to FIG. 3, an explanation of the operation of server 22 is shown in more detail. The user can log onto the server at step 78. A main menu can be displayed at step 80. The main menu can direct the user to lab settings at step 82, user settings at step 84, site settings at step 86, route settings at step 88, device settings at step 90, and report settings at step 91. Lab settings can include such information, designated as 92, as synchronization log, lab location, connection information, and other information. User settings can include such information, designated as 94, as user name, password, and role. The role of a user can include: administrators which connect to portable device 14 or server 22 and have full access; couriers which can access portable device applications, run reports, review synchronization logs, and receive announcements from server 22; client service representative (CSR) which is a service representative for particular sites; report viewers which allow these users to review reports; route managers which are given, generally, full access to portable device 14 and server 22, with the exception of modifying lab settings and site users which allow individuals to view specimen information, but restricted to only those sites associated with the site user. Contact information for user can also be accessed under user setting designated as 96. Site settings can include the site name, address, family name, customer service rep name, signature status, destination point, pickup site, and notes designated as 98 as well as action by those designated as 100. Action items can include notes, messages or other information to be transmitted to portable device 14 for review by the user of the portable device. Route settings can include route name, description, site information, and estimated times for the medical courier to arrive at the particular site in that route. This information is designated generally as 102. Device information can include the name, the number, the date the device was added to the system, and whether the device is enabled, or not. This information is designated generally as 104. Reporting settings can include such information, designated as 105, as site listing reports, destination point reports, specimen alert reports, daily courier metrics reports, courier visit reports, total reports, courier summary reports and data master reports.

Further, inventory information concerning supplies for particular sites can be stored in either service computer readable medium 26 or the portable computer readable medium 16. Information can then be maintained at portable device 14 so that the medical courier can take inventory at a particular site, drop off inventory, and maintain information for the inventory for a particular site. Such information concerning inventory can include historical records for inventory requests, inventory fulfillments, and comparison of inventory use for particular specimens received from the particular site. Since portable device 14 is in communication with server 22, information stored on portable device computer readable medium 16 can be received by server 20 and stored on computer readable medium 26. This would allow a user to access server 22 through network 24 using a terminal 36 and be provided with reports and information concerning inventory and specimen status. For example, a collection site user could access terminal 36 and discover when and what specimens were delivered to what lab location. Further, the lab location could also access server 22 through terminal 36 that would allow the lab to update the status of the specimen. Such information associated with the specimen could include the date the specimen was tested, the date the specimen was received, the medical courier who delivered it, the time and date it was delivered, as well as even attach the lab report itself and associate such information to the specimen.

Information that can be transmitted to portable device 14 for the medical courier to include is a checklist to ensure proper procedures are followed by the medical courier. Information that can be included in such a checklist can include that the courier must acknowledge that all receipts and deliveries have been completed, supplies have been accounted for, and the specific sites and lab locations of a route have been visited.

In one embodiment, a photograph can be taken and entered into the portable device to provide further process information associated with the site, the specimen, or the lab location. A camera, digital or otherwise, can be connected to portable device 14.

When a specimen is delivered to a lab, the portable device 14 can also be used to track the specimen within the lab at various points during the testing procedure. This can allow for the medical specimen to be associated with specific tasks and locations within the lab to provide even more information concerning chain of custody, testing process, and status of the medical specimen. The lab location can also enter the specimen identification mark, such as by scanning the specimen, and since the lab can have access to server 22 in computer readable medium 26, specimen information, even including the testing information, can be transmitted and recorded by server 22, for a complete and comprehensive collection, tracking, and management of information concerning the receipt, delivery, testing, and results of the medical specimen. This information can be accessed by the user of the collection location to even receive the report information from the testing location. The invention may be better understood by reviewing the attached user manual herein fully incorporated by reference.

While specific illustrations of the invention have been used to those skilled in the art, this invention and this disclosure is not intended to limit the scope of the following claims in any manner.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. A system for tracking a specimen wherein a specimen courier uses a portable computing device comprising:
a portable computer readable medium; and
a set of portable computer readable instructions embodied in said portable computer readable medium for receiving specimen identification information from said computer readable medium, receiving process information from said compute readable medium representing that said specimen is ready for pick up from said first location, receiving process information from the courier that said specimen has been picked up from said first location, transmitting said process information to said computer readable medium representing that said specimen has been picked up, receiving process information from the courier representing that said specimen has been delivered to said second location, and transmitting said process information to said computer readable medium representing that said specimen has been delivered to said second location.

13. The system of claim 12 wherein said computer readable instructions include instructions for generating a chain of custody report representing the status of said specimen at particular times and at particular locations.

14. The system of claim 12 wherein said computer readable instructions includes instructions for generating a drop-off list representing that said specimen is to be delivered to said second location.

15. (canceled)

16. (canceled)

17. (canceled)

18. (canceled)

19. (canceled)

20. (canceled)

21. (canceled)

22. (canceled)

23. The system of claim 12 wherein said computer readable instructions include instructions for receiving a plurality of specimen identification information, receiving a plurality of first location information wherein each specimen identification information is associated with a first location information, receiving progress information for each one of said plurality of specimen information indicating that said specimens are ready for pickup, and determining a route between said first plurality of locations so that said plurality of specimens can be picked up according to said route.

24. A method of tracking a specimen having identification information and associated status information originating from a first location having first location information for processing at a second location having second location information, said method comprising the steps of:
receiving specimen identification information;
associating a first location information with said specimen identification information;
picking up a specimen from a first location;
updating status information indicating that said specimen has been picked up from said first location;
delivering specimen to a second location for processing;
associating second location information with said specimen identification information; and
updating said status information indicating that said specimen has been delivered to said second location.

25. The method of claim 24 including the steps of comparing said status information with said specimen identification information located on said specimen to determine if the specimen is the correct specimen to pick up.

26. The method of claim 24 including the steps of reviewing said status information to determine if said specimen has been delivered to said second location.

27. The method of claim 24 including the steps of comparing said status information with the physical location of said specimen to determine if said specimen is at the correct location.

28. The method of claim 24 including the steps of comparing said status information with the physical location of said specimen to determine if said status information currently reflects the physical location of said specimen.
